(19) United States
(12) Patent Application Publication
(10) Pub. No.: US 2024/0177818 A1
(43) Pub. Date: May 30, 2024

(54) METHODS AND SYSTEMS FOR SUMMARIZING DENSELY ANNOTATED MEDICAL REPORTS

(71) Applicant: GE Precision Healthcare LLC, Wauwatosa, WI (US)

(72) Inventors: Shivappa Goravar, Bengaluru (IN); Akshit Achara, Bengaluru (IN); Sanand Sasidharan, Bengaluru (IN); Anuradha Kanamarlapudi, Bengaluru (IN)

(21) Appl. No.: 18/059,890

(22) Filed: Nov. 29, 2022

(51) Int. Cl. G16H 15/00; G06F 40/295; G16H 50/70

(52) U.S. Cl. CPC G16H 15/00; G06F 40/295; G16H 50/70

(57) ABSTRACT

Various methods and systems are provided for generating and displaying summaries of patient information extracted from one or more medical reports stored in an electronic medical record (EMR) of a patient. In one embodiment, a method for summarizing medical reports includes, receiving a medical report for a patient, classifying the medical report into a category of a plurality of pre-determined categories, matching the medical report with an entity recognition model from a library of entity recognition models based on the category, identifying a plurality of named entities in the medical report using the entity recognition model, refining the plurality of named entities to produce a summary of the medical report, and displaying the summary of the medical report via a display device.

### FIELD

Embodiments of the subject matter disclosed herein relate to patient information, and more particularly to automatically identifying and summarizing relevant patient information.

### BACKGROUND

Digital collection, processing, storage, and retrieval of patient medical records may include a conglomeration of large quantities of data. In some examples, the data may include numerous medical procedures and records generated during investigations of the patient, including a variety of examinations, such as blood tests, urine tests, pathology reports, image-based scans, etc. Duration of the diagnosis of a medical condition of a subject followed by treatment may be spread over time from few days to few months or even years in the case of chronic diseases such as cancer or diabetes. Over the course of diagnosing and treating chronic disease, the patient may undergo many different treatments and procedures, and/or may move to different hospitals and/or geographic locations.

Physicians are increasingly relying on Electronic Medical Record (EMR) systems to evaluate the medical history of the patient during diagnosis, treatment, and monitoring of a patient's condition. For patients with chronic illnesses, there are often hundreds or even thousands of EMRs resulting from numerous visits. Sorting and extracting information from past EMRs for such patients is a slow and inefficient process, increasing a likelihood of missing records with relevant data which may be spread out across a large number of less informative routine visit records.

Further, approaches for automatically extracting information from EMRs using machine learning systems perform poorly when a number of classes of the medical observations is large, as informative but rare classes of medical observations (e.g., observation of metastasis) may be obscured by less informative but more common classes of medical observations, as the machine learning system may learn to prioritize identification of the more commonly occurring but less informative classes of medical observations. A probability of detecting rare but informative medical observations in EMRs using machine learning systems may be further reduced in situations where more frequent but less informative medical observations occur in a same EMR as rarer but more informative medical events, such as may be the case in densely annotated EMRs with a high density of reported medical events/observations. Therefore, it is generally desired to explore approaches for automatically summarizing medical reports, which accurately detect sparsely represented but informative medical observations/events, even when co-occurring with more robustly represented but less informative medical observations/events within a single EMR.

### BRIEF DESCRIPTION

In one embodiment, the disclosure provides a method for summarizing medical reports comprising, receiving a medical report for a patient, classifying the medical report into a category of a plurality of pre-determined categories, matching the medical report with an entity recognition model from a library of entity recognition models based on the category, identifying a plurality of named entities in the medical report using the entity recognition model, refining the plurality of named entities to produce a summary of the medical report, and displaying the summary of the medical report via a display device. In this way, named entities (e.g., classes of medical observations/events) may be identified by an entity recognition model trained for the category of medical report to which the currently evaluated medical report belongs. In this way, medical reports may be summarized by an entity recognition model trained to account for frequency/representation imbalances of the entity classes in the training dataset, thereby reducing a probability of rare but informative medical observations being omitted from a medical report summary.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 illustrates a system for summarizing and displaying clinical information of a patient to a user in accordance with an aspect of the disclosure;

FIG. 2 shows a block diagram schematically illustrating a flow of data when generating patient information summaries using a plurality of trained models, according to an embodiment of the disclosure;

FIG. 3 shows a flowchart illustrating a high-level method for generating patient information summaries using a plurality of trained models, according to an embodiment of the disclosure;

FIG. 4 shows a block diagram schematically illustrating a training system for training a plurality of models to recognize entities in text data, according to an embodiment of the disclosure;

FIG. 5 shows a flowchart illustrating an exemplary method for training a plurality of models to recognize entities in text data, according to an embodiment of the disclosure;

FIG. 6 shows a flowchart illustrating an exemplary method for mapping a medical report to a list of entity classifications using an entity recognition model, according to an embodiment of the disclosure;

FIG. 7 shows a flowchart illustrating a first exemplary method for adjusting a base loss for an entity classification based on a plurality of training parameters to produce a list of adjusted losses, according to an embodiment of the disclosure;

FIG. 8 shows one example of determining a base loss for an entity classification vector and scaling the base loss by a loss adjustment factor corresponding to a ground truth annotation, according to the first exemplary method of FIG. 7;

FIG. 9 shows a flowchart illustrating a second exemplary method for adjusting a base loss for an entity classification based on a plurality of training parameters to produce a list of adjusted losses, according to an embodiment of the disclosure;

FIG. 10 shows one example of determining a base loss for each of a plurality of entity classification scores of an entity classification vector, and scaling each base loss by a corresponding loss adjustment factor, according to the second exemplary method of FIG. 9;

FIG. 11 shows a flowchart illustrating a first exemplary method for classifying a medical report into one of a plurality of pre-determined categories, according to an embodiment of the disclosure;

FIG. 12 shows a flowchart illustrating a second exemplary method for classifying a medical report into one of a plurality of pre-determined categories, according to an embodiment of the disclosure;

FIG. 13 is a first excerpt of an example output of a system for summarizing clinical information of a patient, according to an embodiment of the disclosure;

FIG. 14 is an example display of the example output of FIG. 13, according to an embodiment of the disclosure;

FIG. 15 is a second excerpt of an example output of a system for summarizing clinical information of a patient, according to an embodiment of the disclosure;

FIG. 16 is an example display of the example output of FIG. 15, according to an embodiment of the disclosure.

### DETAILED DESCRIPTION

The following description relates to various embodiments of methods and systems to summarize information within an electronic medical record (EMR) of a patient, by detecting named entities that are informative to doctors in digitized medical reports of the EMR, and generating a summary of patient information relating to the named entities. The summary may be formatted in various ways and may be customized, depending on implementation. By generating the patient summary, an amount of time spent by a caregiver reviewing medical reports included in the EMR may be reduced, thereby freeing the caregiver up to address other tasks. Additionally, an amount of relevant patient information made available to the caregiver during the caregiver's limited time spent reviewing the medical reports may be increased, resulting in improved patient outcomes.

A category of named entities, (also referred to herein as a class of named entities, a named entity category, a named entity class, or other similar terms) may be a classification, categorization, or label associated with a text expression (e.g., a word or series of words) found in a medical report included in an EMR. For example, “diseases” may be a first category of named entity including terms such as “cancer”, “hepatitis”, “lewy body dementia”, and “multiple sclerosis”. “Anatomy” may be a second category of named entity including terms such as “heart”, “anterior cruciate ligament”, and “C4 vertabrae”. An entity recognition model may be trained to label words of a text document as belonging to one or more, or none, of the categories of named entities. Although generally sharing similar semantic qualities, various categories of named entities may be defined, for example, by a doctor, a group of doctors, a medical association, hospital administrators, or other healthcare professionals. In some embodiments, the categories of named entities may be organized in a hierarchical manner with super-categories and sub-categories. For example, “disease” may be a category of named entities, which includes a sub-category of named entities relating to cancer (e.g., the sub-category may be given the designation “cancer”). The “cancer” sub-category of named entities may in turn include a sub-category for diseases pertaining to lung cancer (e.g., the sub-category of named entities may be given the designation “lung cancer”); and so on. The categories of named entities may be predefined (e.g., defined via the annotations of the training dataset), and/or may be periodically added to or changed. For example, a new category or sub-category of an entity may be added.

Identifying named entities from medical reports can not only help in diagnosis and treatment but also save a lot of manual effort and time. Deep learning models like BERT (Bidirectional Encoder Representations from Transformers) and Bi-LSTM based models have shown promising results. In conventional approaches, to achieve good performance on named entity recognition (NER), a deep learning model should be trained on labelled medical reports and other relevant data. EMR's are densely annotated, indicating large number of words in each report are clinically relevant and may therefore be informative to a medical professional when reviewing a patient's medical history. Training a model and achieving good accuracy across multiple categories of named entities conventionally requires a large amount of annotated data for each named entity category. Due to the nature of medical reports, the frequency of each category of named entities present in each type of report will vary significantly. Addition of new reports to the training corpus still may not solve the class imbalance between the different categories of named entities. As a result, despite increasing an amount of training data, the categories of named entities may remain skewed/imbalanced. A classification model trained with an imbalanced dataset may prioritize the more robustly represented classes of named entities, while performing poorly on (and potentially mis-labeling) less frequent categories of named entity present in medical reports.

The inventors herein disclose methods and systems which may at least partially address the above issues. In one example, a method employing an adaptive penalty/loss for each category of named entities may increase a probability of a deep learning model accurately identifying words belonging to a named entity category of interest, or an under-represented category of named entities. Using an adaptive loss based approach which retains all categories of named entities in the training data, will help to preserve the context of the sentences/medical report, but still enable tuning of the deep learning model for specific named entity classes of interest. The herein disclosed methods and systems may include training a plurality of entity recognition models, wherein each model uses a distinct set of training parameters specifically set to account for named entity classes of interest and/or named entity class imbalances in a particular category of medical report. Thus, a library of trained entity recognition models may be generated, wherein at inference time a medical report may be matched to an entity recognition model based on attributes of the medical report, wherein the entity recognition model specializes in identifying named entities in medical reports possessing said attributes.

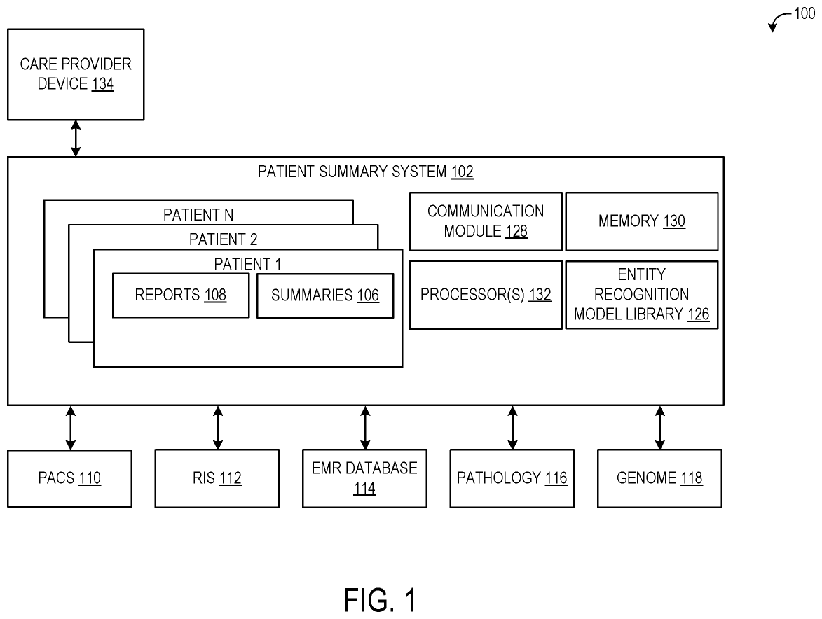

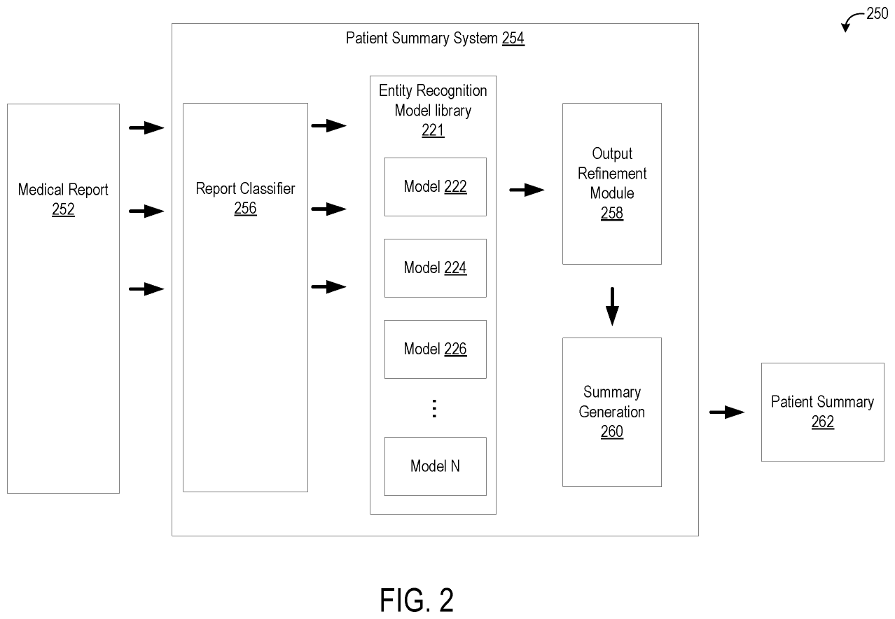

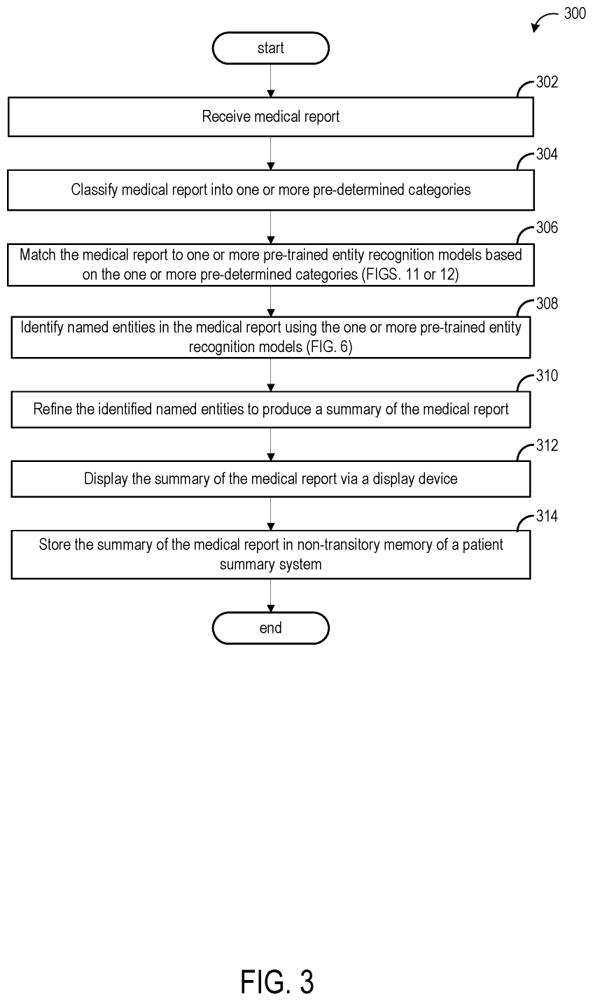

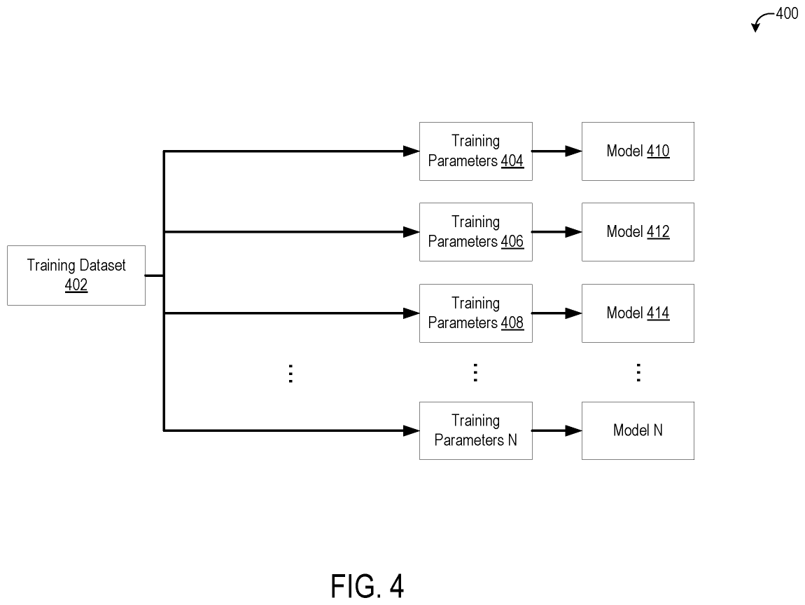

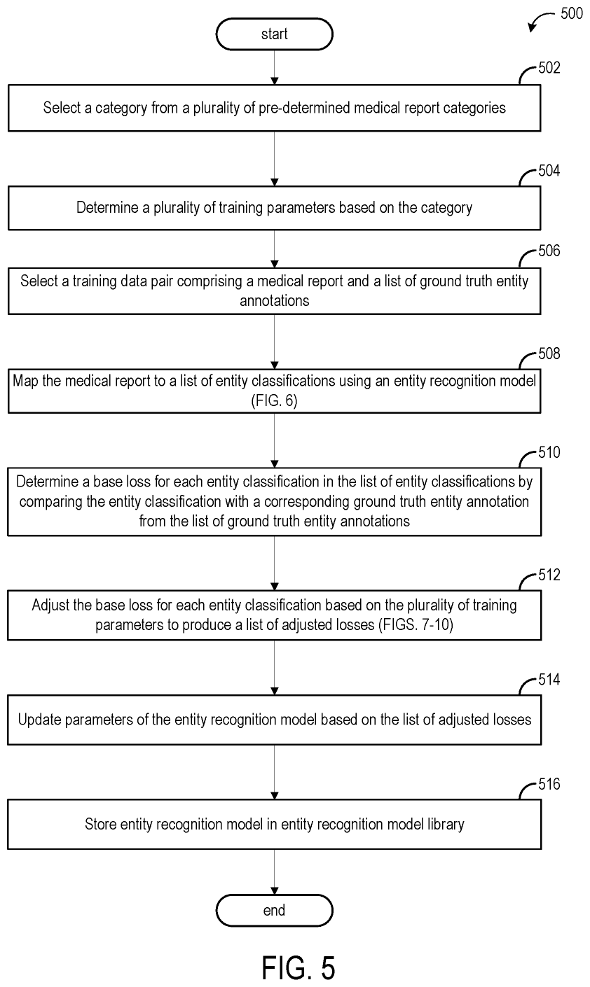

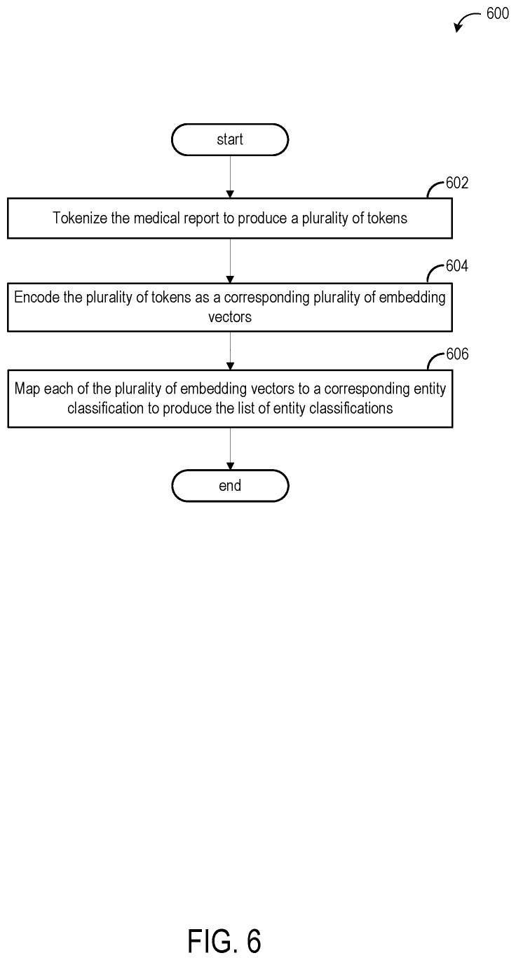

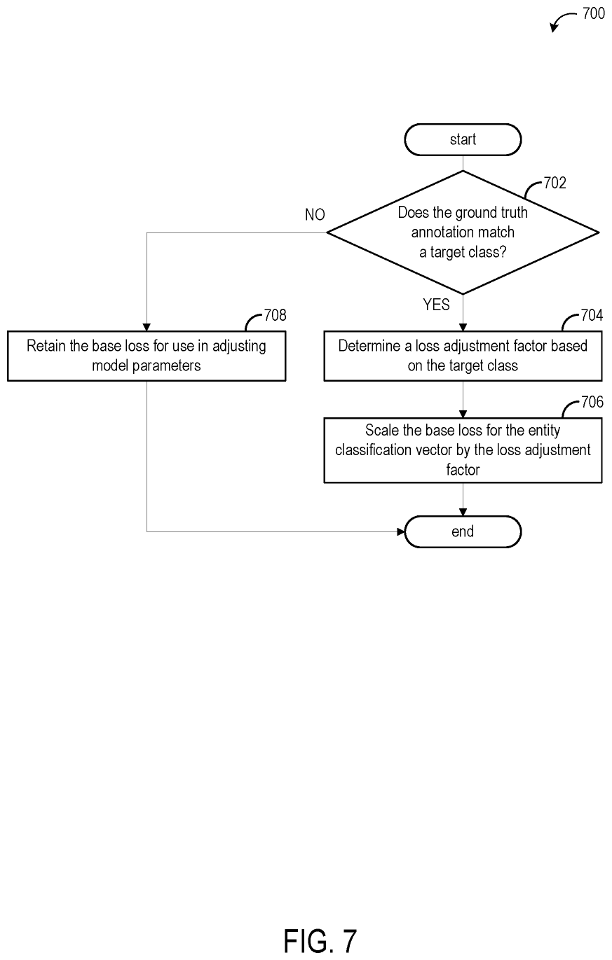

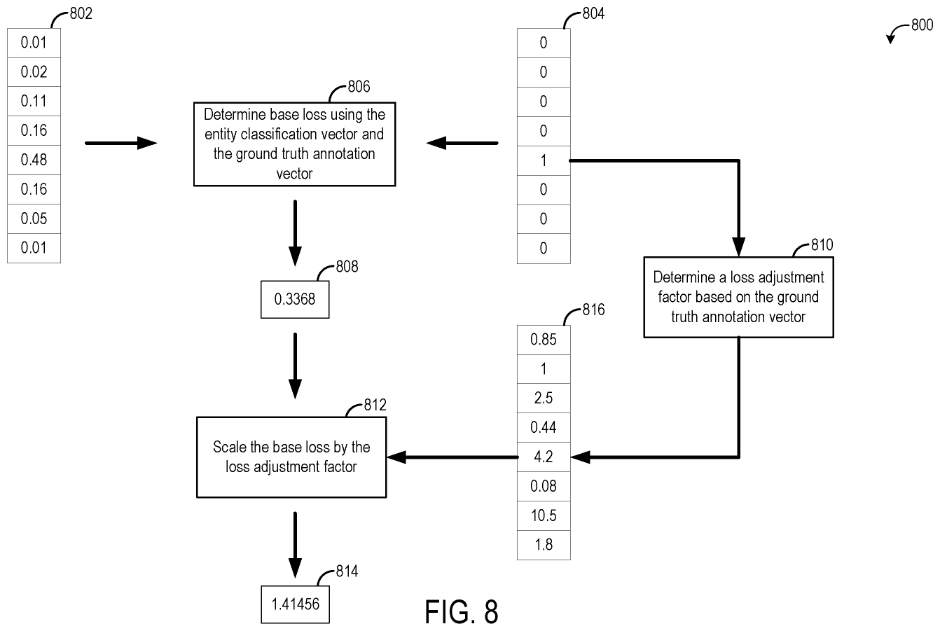

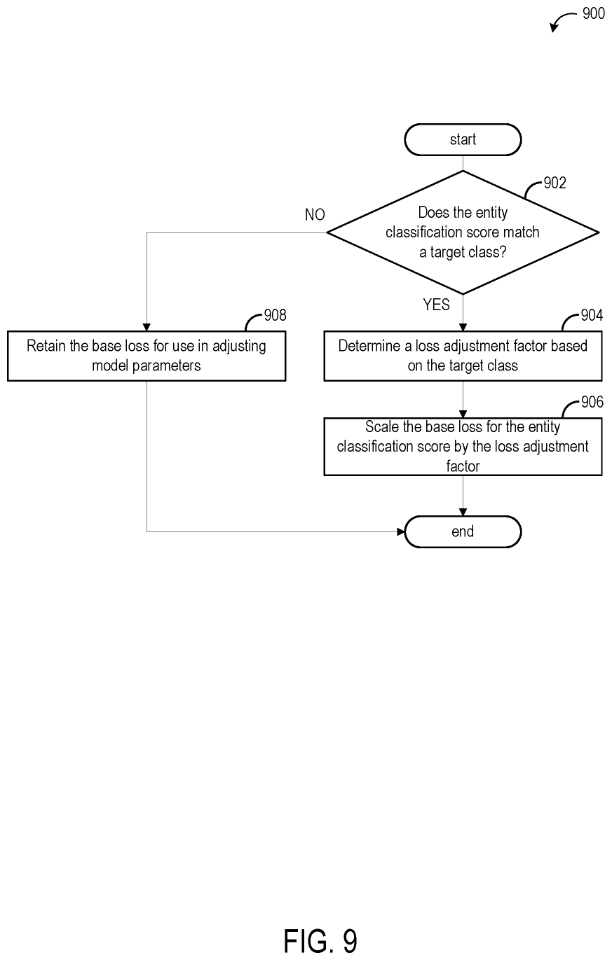

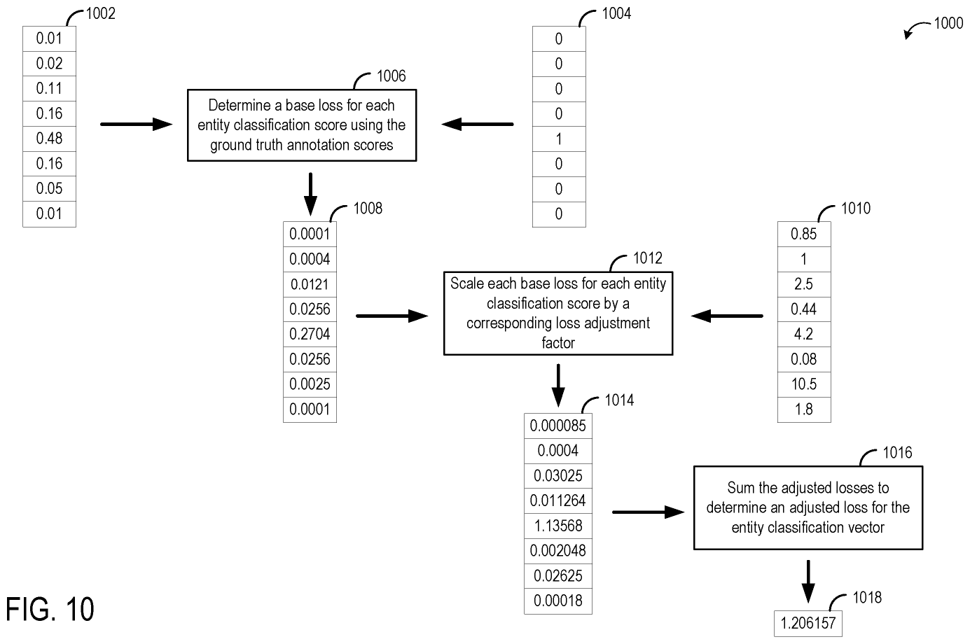

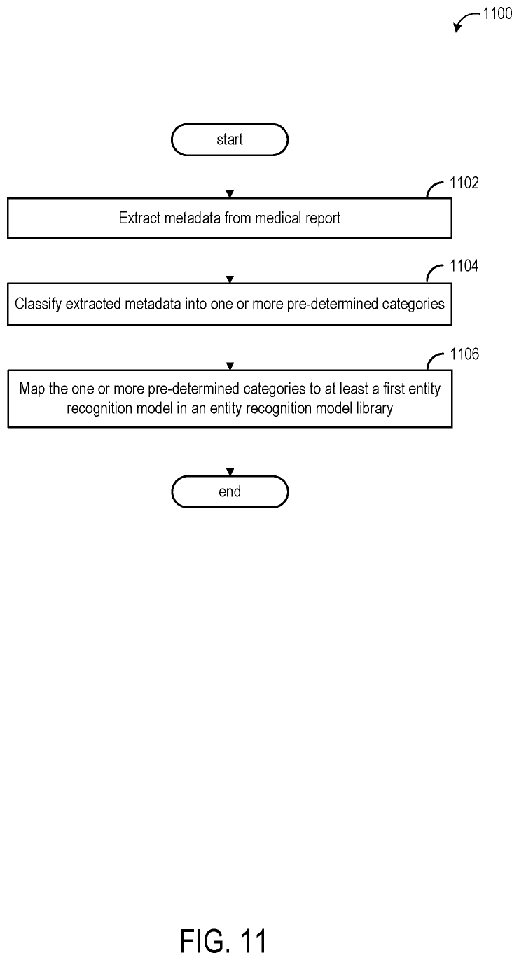

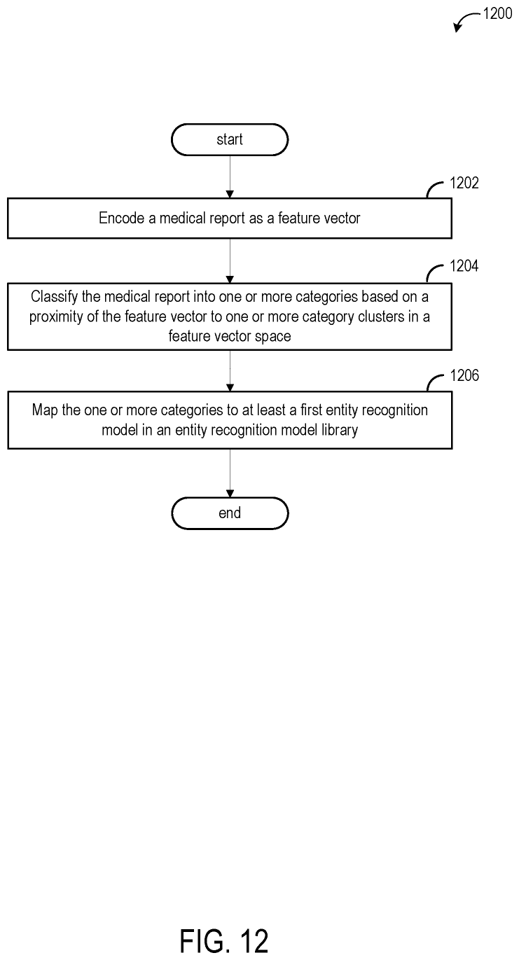

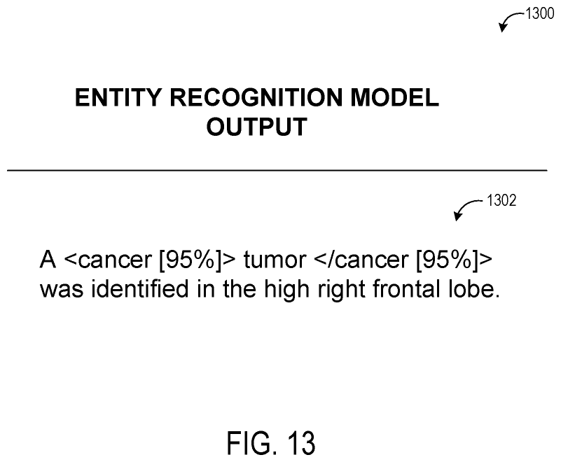

An example patient information system is shown in FIG. 1, which may include a plurality of entity recognition models stored in an entity recognition model library, used to generate a patient information summary from medical reports. The patient information system shown in FIG. 1 may be used to summarize one or more medical reports of a patient, as illustrated in FIG. 2, according to one or more of the operations of method 300 shown in FIG. 3. The plurality of entity recognition models employed by the patient information system may be trained using a single dataset, but with distinct training parameters particular to distinct categories of medical reports, as illustrated in FIG. 4 according to one or more of the operations of method 500 depicted in FIG. 5. In one embodiment, the patient information system may classify a medical report according to one or more operations of method 1100, shown in FIG. 11, or method 1200, shown in FIG. 12, and may select a trained entity recognition model from a library of trained entity recognition models based on the classification of the medical report. The entity recognition models in the entity recognition model library may be trained to accurately identify informative medical events/observations described in a medical report using an adaptive loss strategy, such as the adaptive loss strategy illustrated in FIG. 8 which may be performed by executing one or more operations of method 700 shown in FIG. 7, or in another embodiment by performing the adaptive loss strategy illustrated in FIG. 10 which may be performed by executing one or more operations of method 900 shown in FIG. 9. The entity recognition models may transduce a medical report into a sequence of embedding vectors, and then classify each of the sequence of embedding vectors into one or more, or none, of the named entity classes, by executing one or more of the operations described in method 600 shown in FIG. 6. The identified named entities may be used to generate a summary of the medical history of a patient. FIGS. 13, 14, 15, and 16 provide examples of patient summaries which may be displayed to a user, wherein one or more named entities may be visually emphasized along with an optional label of the named entity class.

Embodiments of the present disclosure will now be described, by way of example, with reference to the figures, in which FIG. 1 schematically shows an example patient information system 100 that may be implemented in medical facility such as a hospital. Patient information system 100 may include a patient summary system 102. Summary system 102 may include resources (e.g., memory 130, processor(s) 132) that may be allocated to generate and store patient summaries for one or more medical reports drawn from one or more EMRs for each of a plurality of patients. For example, as shown in FIG. 1, summaries 106 and optionally medical reports 108 are stored on summary system 102 for a first patient (patient 1); a plurality of additional summaries and medical reports may be stored on and/or generated by summary system 102, each corresponding to a respective patient (patient 2 up to patient N).

Each summary of summaries 106 may include text and/or graphical representations of pertinent/relevant patient information associated with entities included in a given medical report. The entity-related information included in summaries 106 may include information related to disease, tissue, anatomy, problem, test, treatment, and/or other information included in the medical report and identified as being of interest.

The patient information that is presented via summaries 106 may be stored in different medical databases or storage systems in communication with summary system 102. For example, as shown, the summary system 102 may be in communication with a picture archiving and communication system (PACS) 110, a radiology information system (RIS) 112, an EMR database 114, a pathology database 116, and a genome database 118. PACS 110 may store medical images and associated reports (e.g., clinician findings), such as ultrasound images, MRI images, etc. PACS 110 may store images and communicate according to the DICOM format. RIS 112 may store radiology images and associated reports, such as CT images, X-ray images, etc. EMR database 114 store electronic medical records for a plurality of patients. EMR database 114 may be a database stored in a mass storage device configured to communicate with secure channels (e.g., HTTPS and TLS), and store data in encrypted form. Further, the EMR database is configured to control access to patient electronic medical records such that only authorized healthcare providers may edit and access the electronic medical records. An EMR for a patient may include patient demographic information, family medical history, past medical history, lifestyle information, preexisting medical conditions, current medications, allergies, surgical history, past medical screenings and procedures, past hospitalizations and visits, etc. Pathology database 116 may store pathology images and related reports, which may include visible light or fluorescence images of tissue, such as immunohistochemistry (IHC) images. Genome database 118 may store patient genotypes (e.g., of tumors) and/or other tested biomarkers.

When requested, a summary from summaries 106 may be displayed on one or more display devices, such as a care provider device 134, and in some examples more than one care provider device, may be communicatively coupled to summary system 102. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. The care provider devices may be located locally at the medical facility (such as in the room of a patient or a clinician's office) and/or remotely from the medical facility (such as a care provider's mobile device).

When viewing a summary of summaries 106 via a display of a care provider device, a care provider may enter an input (e.g., via the user input device, which may include a keyboard, mouse, microphone, touch screen, stylus, or other device) that may be processed by the care provider device and sent to the summary system 102. The user input may trigger display of the medical report that is summarized by a summary in summaries 106, trigger progression to a prior or future summary, trigger updates to the configuration of the summary, or other actions.

To generate the summaries 106, summary system 102 may include an entity recognition model library 126, comprising a plurality of entity recognition models. Each entity recognition model stored in the entity recognition model library 126 may be a machine learning model, such as a deep neural network, trained to recognize one or more categories of named entities within a particular category of medical report of a patient. For example, a first entity recognition model stored in entity recognition model library 126 may be trained to identify named entities in a plurality of named entity categories in MRI reports, which may be received from EMR database 114 or PACS 110; a second named entity recognition model stored in entity recognition model library 126 may be trained to identify named entities in the same plurality of named entity categories, but within pathology reports; and a third entity recognition model stored in the entity recognition model library 126 may be trained to identify named entities in the plurality of named entity categories in operative reports; and so on.

To generate a summary, a medical report may be classified into one of a pre-determined number of medical report categories, before being assigned to a named entity recognition model in the named entity recognition model library 126 which was trained using training parameters set based on the particular medical report category, e.g., the training parameters may be set adjust a loss for various target classes of named entities to account for class imbalances within the particular medical report category, as well as to prioritize identification of named entities which are deemed informative within the context of the particular category of medical report.

In various embodiments, the entity recognition model from the entity recognition model library may also output probabilities associated with the one or more named entities identified in the medical report. In some embodiments, each entity classification may comprise an entity classification vector, comprising a plurality of entity classification scores, wherein each entity classification score may indicates a probability of the given named entity belonging to one of a plurality of pre-determined categories of named entities. As an example, an entity recognition model trained to identify named entities in the classes of “disease”, “anatomy”, “drug”, and “outside” (where “outside” is an umbrella class covering all named entities that do not fall into one of the other designated classes) may produce an entity classification vector for the phrase “non-small cell lung carcinoma” such as [0.7, 0.2, 0.02, 0.08], thus indicating a 70% probability of the named entity belonging to the “disease” class of named entities, a 20% probability of the named entity belonging to the “anatomy” class of named entities, and a 2% and 8% probability of the named entity belonging to the “drug” or “outside” class of named entities, respectively.

The named entities identified by the named entity recognition model library may be further refined (e.g., duplicates and less informative named entities removed, an order of the remaining named entities adjusted for clarity, etc.), to produce a summary of the medical report.

Summary system 102 includes a communication module 128, memory 130, and processor(s) 132 to store and generate the summaries, as well as send and receive communications, graphical user interfaces, medical data, and other information.

Communication module 128 facilitates transmission of electronic data within and/or among one or more systems. Communication via communication module 128 can be implemented using one or more protocols. In some examples, communication via communication module 128 occurs according to one or more standards (e.g., Digital Imaging and Communications in Medicine (DICOM), Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), etc. using any past, present, or future communication protocol (e.g., BLUETOOTH™, USB 2.0, USB 3.0, etc.).

Memory 130 one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

One or more of the devices described herein may be implemented over a cloud or other computer network. For example, summary system 102 is shown in FIG. 1 as constituting a single entity, but it is to be understood that summary system 102 may be distributed across multiple devices, such as across multiple servers. Further, while the elements of FIG. 1 are shown as being housed at a single medical facility, it is to be appreciated that any of the components described herein (e.g., EMR database, RIS, PACS, etc.) may be located off-site or remote from the summary system 102. Further, the longitudinal data utilized by the summary system 102 for the summary generation and other tasks described below could come from systems within the medical facility or obtained through electronic means (e.g., over a network) from other referring institutions.

While not specifically shown in FIG. 1, additional devices described herein (e.g., care provider device 134) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on summary system 102 or sent via summary system 102. For example, care provider device 134 may store a user interface template for a patient timeline that a user of care provider device 134 may configure with placeholders for desired patient information. When the summary is displayed on the care provider device, the relevant patient information may be retrieved from summary system 102 and inserted in the placeholders. The user input devices may include keyboards, mice, touch screens, microphones, or other suitable devices.

FIG. 2 shows a block diagram 250 schematically illustrating a flow of data when generating a patient information summary from a medical report using the trained entity recognition models stored in the entity recognition model library 221 of FIG. 2. (e.g., model 222, model 224, model 226, up to an N^{th} model). Block diagram 250 includes a medical report 252, which may be processed by a patient summary system 254 (e.g., patient summary system 102) to generate a patient summary 262. Patient summary system 254 may include an output refinement module 258, and a summary generation block 260, which may represent modules or portions of code of patient summary system 254 and/or processing stages including executing the portions of code and receiving input from human users of patient summary system 254.

Patient summary system 254 may classify an incoming medical report into one of a pre-determined number of medical report categories, using report classifier 256. In some embodiments, classifier 256 may evaluate metadata associated with a medical report to assign a medical report classification to a medical report. In some embodiments, the report classifier 256 may encode the medical report as a feature vector, using one or more of the text and metadata of the incoming medical report, and assign the medical report to a medical report category based on the feature vector. In one example, the pre-determined medical report categories may be encoded in a feature vector space as clusters, and the medical report may be assigned to one or more of the pre-determined medical report categories based on a proximity of the feature vector of the report to one or more of the clusters within the feature vector space.

Once the medical report has been classified into a medical report category, an entity recognition model stored in entity recognition model library 221 may be matched with the medical report based on the medical report category. As shown in FIG. 2, N distinct entity recognition models, including entity recognition model 222, entity recognition model 224, and entity recognition model 226, may be stored in entity recognition model library 221, wherein N is a positive integer greater than one. In some embodiments, entity recognition models may be indexed within the entity recognition model library 221 based on the medical report category for which their training parameters were set.

The medical report may be input into the entity recognition model corresponding to the medical report category. The entity recognition model may output a plurality of named entity classifications for each token of the medical report. In some embodiments, the entity recognition model may output a version of medical report 252, with identified named entities visually emphasized or otherwise demarcated.

Outputs of the entity recognition model may then be refined using the output refinement module 258. In some embodiments, output refinement module 258 may remove duplicate identified named entities (e.g., if “lung cancer” has been identified several times within a single report, duplicate instances of “lung cancer” may be removed in order to streamline the report summary). In some embodiments, the output refinement module 258 may prioritize or group identified named entities, e.g., by re-ordering identified named entities by hard coded rules regarding contextual importance of particular named entities within the medical report category. In some embodiments, the output refinement module 258 may include adjusting or changing one or more entity labels based on context-based clinical knowledge and/or natural language processing (NLP), as described in greater detail below.

After refining the identified named entities, a patient summary 262 may be generated by summary generation block 260 of patient summary system 254. In some embodiments, the summary generation block produces a formatted version of excerpts of the medical report, such as those discussed below with reference to FIGS. 13-16, based on identified and refined named entities output by output refinement module 258. In some embodiments, the summary generation block may insert one or more of the identified and refined named entities into a template summary, wherein the one or more identified and refined named entities may be inserted in a pre-determined order. In one example, a summary template may comprise portions of text and one or more insertion flags, wherein said insertion flags indicate a point of insertion for one or more identified and refinement name entities, if present. As an example, a template summary may read “[medical report category] summary for patient [patient name]: [disease] was found on [report date] via [medical imaging type] within [anatomy]”, wherein portions within “[ ]” indicate insertion flags for named entity categories described within the insertion flags/brackets. An example of the above template completed by insertion of identified and refined name entities may read “MRI report summary for John Doe: Edema was found on Nov. 20, 2020 via T1 weighted multi-planar scanning within the supraspinatus”.

Referring to FIG. 3, a flowchart illustrating method 300, which may be executed by a patient information system, such as patient information system 100, to obtain and summarize medical reports of a patient using one or more entity recognition models. Medical report summaries generated by method 300 may be transmitted to a device, such as a device of a care provider, and displayed via a display device, and/or the medical report summary may be stored for future retrieval, such as by storing a patient medical report summary in the patient information system 100.

Method 300 begins at operation 302, wherein the patient information system receives a medical report from one or more EMR databases or other patient information data storage systems communicatively coupled to the patient information system. In some embodiments, the patient information system may access one or more patient medical reports based on a request received by a care provider. In some embodiments, a patient information system may pre-emptively summarize a plurality of medical reports of a patient based on inclusion of the patient in a medical report summarization queue.

At operation 304, the patient information system classifies the medical report into one or more pre-determined medical report categories. In some embodiments, the patient information system may access metadata associated with a medical report, such as via a DICOM header of a DICOM formatted medical image, and may classify the medical report based on the accessed metadata. In some embodiments, the medical report may be encoded as a feature vector, and assigned to one or more medical report category clusters in a feature vector space based on proximity of the feature vector encoding of the medical report with the medical report category clusters in the feature vector space.

At operation 306, the patient information system matches the medical report to one or more pre-trained entity recognition models based on the one or more pre-determined categories, as will be described in more detail with reference to FIGS. 11 and 12, below. Briefly, the patient information system may match the medical report with one or more trained entity recognition models based on the medical report category into which the medical report was classified at operation 304. The patient information system may store a plurality of trained entity recognition models in an entity recognition model library, wherein the plurality of models may be indexed according to performance characteristics, training parameters during a training routine for the model, or in some cases with an explicit flag indicating one or more medical report categories for which the trained entity recognition model may be used.

At operation 308, the patient information system identifies named entities in the medical report using the one or more trained entity recognition models. In some embodiments, the selected entity recognition model may identify one or more named entities in the medical report by tokenizing the medical report to produce a plurality of tokens, encoding the plurality of tokens into an embedding space using a deep neural network, such as a transformer based neural network or a recurrent neural network (RNN), to produce a plurality of embedding vectors corresponding to the plurality of tokens, and determining a classification vector for each of the plurality of embedding vectors, wherein a classification vector indicates a probability of the associated text/token belonging to each of a plurality of pre-determined named entity classes.

At operation 310, the patient information system refines the identified named entities to produce a summary of the medical report. In some embodiments, the patient information system may refine the identified named entities by removing duplicate identified named entities (e.g., if “lung cancer” has been identified several times within a single report, duplicate instances of “lung cancer” may be removed in order to streamline the report summary). In some embodiments, patient information system may prioritize or group identified named entities, e.g., by re-ordering identified named entities by hard coded rules regarding contextual importance of particular named entities within the medical report category. In some embodiments, the patient information system may adjust or change one or more entity labels based on context-based clinical knowledge and/or natural language processing (NLP), as described in greater detail below.

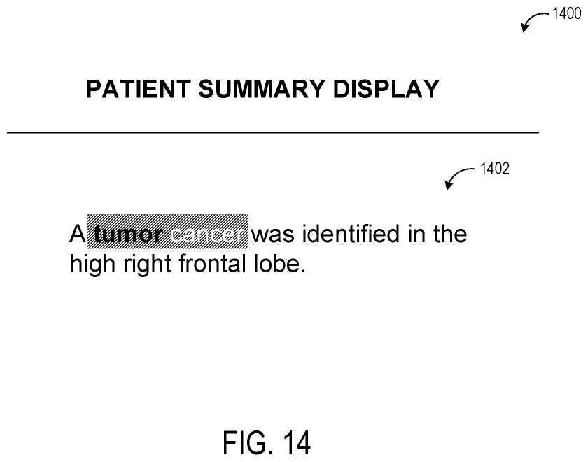

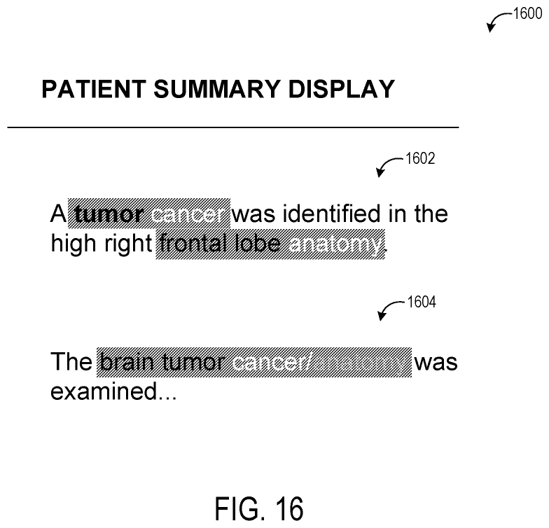

At operation 312, the patient information system displays the summary of the medical report via a display device. Non-limiting examples of summaries which may be displayed are provided in FIGS. 14 and 16, wherein one or more identified named entities are visually emphasized to provide a succinct and informative summary of the medical report received at operation 302.

At operation 314, the patient information system stores the summary of the medical report in non-transitory memory of a patient summary system. Following operation 314, method 300 may end.

In this way, a medical report may be intelligently summarized using an entity recognition model trained bespoke to the medical report category into which the medical report is classified. Thus, a probability of mis-classifying informative named entities present in the medical report is reduced, by utilizing a deep neural network trained using training parameters set to account for skewness and contextual importance of named entities within the medical report category.

Referring to FIG. 4, an illustration of a training strategy 400 for training a plurality of entity recognition models to specialize in named entity identification for distinct medical report categories, while using a same dataset, is shown.

Training strategy 400 includes a single training dataset 402, comprising a plurality of medical reports in various medical report categories, and associated lists of ground truth entity annotation vectors corresponding to the medical reports. The ground truth entity annotation vectors indicate for each token in a corresponding medical report, the category of named entity to which said token belongs (if any). In one example, a ground truth entity annotation vector for named entity classes of “disease”, “anatomy”, “drugs”, and “outside” may be [1, 0, 0, 0], indicating that the associated token or text sequence belongs to the named entity class of “disease” and does not belong to the named entity classes of “anatomy”, “drugs”, or “outside”, as indicated by the associated ground truth entity annotations for each category within the ground truth entity annotation vector.

The training dataset 402 may be used along with N distinct sets of training parameters (that is, training parameters 404, training parameters 406, training parameters 408, up to training parameters N), to train N distinct entity recognition models (that is, entity recognition model 410, entity recognition model 412, entity recognition model 414, up to entity recognition model N, respectively) where N is a positive integer greater than 2. In some embodiments, each set of training parameters (training parameters 404 to N) may be selected based on one or more attributes of one or more medical report categories. As an example, training dataset 402 may comprise N distinct medical report categories, and each set of training parameters may be chosen based on statistical attributes or other features of one of N medical report categories, to produce N distinct entity recognition models, wherein each entity recognition model specializes in identification of named entities for one of the N medical report categories. In some embodiments, the training parameters may include distinct sets of loss adjustment factors for each of a plurality of target classes of named entities. In another example, each of the distinct sets of training parameters may include a unique set of loss adjustment vectors for each of a plurality of named entity classes.

Referring to FIG. 5, an embodiment of a training method 500 for training an entity recognition model to identify named entities in a particular medical report category is shown. Method 500 may be executed by a patient information system, such as patient information system 100, or may be executed by a separate computing system before storing the trained entity recognition models on the patient information system.

Method 500 begins at operation 502, wherein the patient information system selects a category from a plurality of pre-determined medical report categories. In some embodiments, the patient information system may iterate through each of the medical report categories training one or more entity recognition models for each medical report category.

At operation 504, the patient information system determines a plurality of training parameters based on the category. The training parameters may include distinct sets of loss adjustment factors for each of a plurality of target classes of named entities. In another example, each of the distinct sets of training parameters may include a unique set of loss adjustment vectors for each of a plurality of named entity classes. The list of target entities and the associated loss adjustment factors may be selected based on statistical or other attributes of the medical report category selected at operation 502, and further based on domain knowledge of medical experts to prioritize named entities of greater informative value within the context of the medical report category. As an example, the named entity “small cell lung carcinoma” may be sparsely represented with respect to a training dataset, but may be of high informative value within the context of pathology reports, and therefore an associated loss adjustment factor for “non-small cell lung carcinoma” may be determined at operation 504 to account for the under-representation of this named entity within the overall training dataset, as well as to increase a rate of learning of the entity recognition model when identifying training data pairs including the named entity “non-small cell lung carcinoma”. In particular, by setting a loss adjustment factor for “non-small cell lung carcinoma” to a value greater than one, a base loss associated with imperfect classification of this named entity may be increased, causing the entity recognition model to prioritize accurate identification of the named entity. Conversely, named entities which are either over-represented in the training data (particularly when in comparison to a frequency of occurrence of said named entities within the medical report category selected at operation 502), a loss adjustment factor associated with said named entity may be set to a value equal to or less than 1, thereby de-emphasizing the importance of named entity with respect to a rate of learning of the entity recognition model.

At operation 506, the patient information system selects a training data pair comprising a medical report and a list of ground truth entity annotations. The medical report may comprise a string of characters, and the list of ground truth entity annotations include labels/annotations indicating a “correct” named entity category for each of the plurality of tokens of the medical report string. Training data pair selection may be executing according to a training scheduler, wherein in some embodiments each of a plurality of training data pairs may be selected according to a pre-determined sequence, whereas in other embodiments an order or training data pair selection may be randomized to reduce a probability of the entity recognition model learning to anticipate the label of the currently selected training data pair, without learning a more complex representation of the mapping from medical report tokens to named entity classifications.

At operation 508, the patient information system maps the medical report to a list of entity classifications using the entity recognition model (FIG. 6). Turning briefly to FIG. 6, mapping the medical report to a list of entity classifications using the entity recognition model comprises: tokenizing the medical report into a plurality of tokens at operation 602, encoding each of the plurality of tokens into a corresponding embedding vector (e.g., by using a transformer model or other embedding model) at operation 604, and mapping each embedding vector to a corresponding entity classification vector at operation 606, wherein the entity classification vector includes an entity classification score or probability for each class of the named entities.

At operation 510, the patient information system determines a base loss for each entity classification in the list of entity classifications by comparing the entity classification with a corresponding ground truth entity annotation from the list of ground truth entity annotations. Various loss functions may be used to produce the base loss. In some embodiments, a sum of squared errors may be used to determine a base loss for the entity classification vector. In some embodiments, a categorical cross-entropy loss may be employed at operation 510 to determine the base loss of the entity classification vector based on the corresponding ground truth annotation vector.

At operation 512, the patient information system adjusts the base loss for each entity classification based on the plurality of training parameters to produce a list of adjusted losses (FIGS. 7-10 discuss loss adjustment in more detail). In some embodiments, the base loss for each entity classification vector may be scaled based on a list of target entities and further based on an associated loss adjustment factor vector.

At operation 514, the patient information system updates parameters of the entity recognition model based on the list of adjusted losses. In some embodiments, a gradient descent algorithm may be used to determine an approximation of the gradient of the loss function with respect to each trainable parameter of the entity recognition model, and each trainable parameter may be updated by based on said approximated gradient, e.g., by taking subtracting the gradient multiplied by a pre-determined step size from the previous parameter value, in order to generate an updated parameter value.

At operation 516, the patient information system may store the entity recognition model in entity recognition model library. Storage of the entity recognition model in the entity recognition model library may be triggered upon satisfaction of one or more stopping criteria set for the training routine. In one example, achieving below a threshold change in loss between one epoch and the next may satisfy the stopping criteria. In another example, a stopping criteria may be satisfied following a reduction in validation loss below a threshold. In another example, the stopping criteria may be satisfied following a minimization of a validation loss (e.g., a validation loss decreases and then starts to increase with additional training). The entity recognition model may be stored in the entity recognition model library with an index indicating the medical report category for which the entity recognition model was trained, thereby facilitating efficient matching between medical reports and corresponding entity recognition models at inference time. Following operation 516, method 500 may end.

Referring to FIG. 7, a flowchart of an exemplary method 700 of adjusting a base loss based on a medical report category is shown. Method 700 may be executed as part of a training routine of an entity recognition model, such as the training routine described above with reference to FIG. 5.

Method 700 begins at operation 702, wherein the patient information system evaluates if a ground truth annotation of a currently selected training data pair matches a target class for the selected medical report category. If at operation 702 the patient information system determines that the ground truth entity annotation does not match a target class, method 700 may proceed to operation 708, wherein the base loss is retained for the current training data pair. However, if at operation 702 the patient information system determines that the ground truth entity annotation matches a target class for the currently selected medical report category, method 700 proceeds to operation 704.

At operation 704, the patient information system determines a loss adjustment factor based on the target class matching the ground truth entity annotation. In some embodiments, operation 704 may include the patient information system querying a loss adjustment factor vector containing a plurality of loss adjustment factors for a corresponding plurality of target classes selected for a particular medical report category.

At operation 706, the patient information system scales the base loss using the loss adjustment factor determine at operation 704, to produce an adjusted loss for the current training data pair. Following operation 706, method 700 may end. It will be appreciated that method 700 may be executed once during each selection of a training data pair during a training routine of an entity recognition model.

Referring to FIG. 8, an example of the loss adjustment process 800, which may occur as part of execution of method 700, is shown. Loss adjustment process 800 includes operation 806, wherein a base loss 808 for an entity classification vector 802 is determined based on a ground truth entity annotation vector 804. In the example shown by loss adjustment process 800, a sum of squared errors loss function is applied to the entity classification vector and the ground truth entity annotation vector 804.

In parallel with calculation of the base loss 808, operation 810 may be executed, wherein a loss adjustment factor is determined based on the ground truth entity annotation vector 804. In particular, example process 800 depicts selection of a loss adjustment factor from a loss adjustment factor vector 816 based on the ground truth entity annotation given to the current training data pair. The “1” in the 5^{th} row of the ground truth entity annotation vector 804 indicates the current token/text belongs to a 5^{th} class of named entities, and thus a 5^{th} adjustment factor from the loss adjustment factor vector 816 is selected, and used at operation 812 to scale the base loss, producing the adjusted loss 814. As the adjustment factor in example process 800 is 4.2 (that is, greater than 1), the base loss associated with the classification vector 802 will be increased, thereby causing the entity classification model to prioritize learning from this training data pair.

Referring to FIG. 9, a flowchart of an exemplary method 900 of adjusting a base loss based a medical report category is shown. Method 900 may be executed as part of a training routine of an entity recognition model, such as the training routine described above with reference to FIG. 5.

Method 900 begins at operation 902, wherein the patient information system evaluates if an entity classification score matches a target class for the selected medical report category. If at operation 902 the patient information system determines that the entity classification score does not match a target class, method 900 may proceed to operation 908, wherein the base loss is retained for the current training data pair. However, if at operation 902 the patient information system determines that the entity classification score matches a target class for the currently selected medical report category, method 900 proceeds to operation 904.

At operation 904, the patient information system determines a loss adjustment factor based on the target class matching the entity classification score. In some embodiments, operation 904 may include the patient information system querying a loss adjustment factor vector containing a plurality of loss adjustment factors for a corresponding plurality of target classes selected for a particular medical report category.

At operation 906, the patient information system scales the base loss for the entity classification score using the loss adjustment factor determine at operation 904, to produce an adjusted loss for the current training data pair. Following operation 906, method 900 may end. It will be appreciated that method 900 may be executed once during each selection of a training data pair during a training routine of an entity recognition model.

Referring to FIG. 10, an example of the loss adjustment process 1000, which may occur as part of execution of method 900, is shown. Example process 1000 includes determining at operation 1006 a loss vector 1008 comprising the class-wise losses for each of a plurality of named entity classes using an entity classification vector 1002 and a corresponding ground truth entity annotation vector 1004. The entity classification vector 1002 comprises a plurality of entity classification scores for a pre-determined plurality of named entity classes, wherein, in the example shown by process 1000, each entity classification score indicates a probability of the currently evaluated token/text belonging to a named entity class associated with the current row (e.g., the probability given by the 2^{nd} row of the entity classification vector indicates a probability of the current token belonging to a 2^{nd} class of the plurality of named entity classes).

The loss vector 1008 may be adjusted at operation 1012 using loss adjustment factor vector 1010. As shown in example process 1000, the loss adjustment vector comprises a plurality of loss adjustment factors corresponding to the rows/classes of the entity classification vector 1002. The base loss in each row of base loss vector 1008 is scaled/adjusted by a corresponding loss adjustment factor from the loss adjustment factor vector 1010 (e.g., the base loss in the 3^{rd} row of base loss vector 1008 is adjusted by the loss adjustment factor in the 3^{rd} row of the loss adjustment factor vector 1010). Adjusting the base loss vector 1008 using the loss adjustment factor vector 1010 at operation 1012 produces adjusted loss vector 1014. Summing the adjusted losses in the adjusted loss vector 1014 at operation 1016 produces a single adjusted loss 1018 for the current entity classification vector 1002. In some embodiments, operation 1012 and operation 1016 may be combined into a single step, wherein the adjusted loss 1018 for the entity classification vector 1002 is produced by taking the dot product of the base loss vector 1008 and the loss adjustment factor vector 1010.

Referring to FIG. 11, a flowchart of a first embodiment of a method 1100 for classifying a medical report into one of a pre-determined number of medical report categories is shown. Method 1100 may be executed by a patient information system, such as patient information system 100, or may be executed by a separate computing system before transmitting the medical report classification to the patient information system. Method 1100 may be executed alone, or as part of another method herein disclosed, such as at operation 306 of method 300, in order to determine a category or classification to which a currently evaluated medical report belongs. In some embodiments, the pre-determined number of medical report categories may comprise a finite number of categories, selected based on domain knowledge (e.g., a first medical report category may be created for reports regarding diagnostic X-ray images of lungs, whereas a second medical report category may be created for reports regarding MRI images of brain). However, in some embodiments, the pre-determined number of medical report categories may be chosen based linguistic and/or statistical grouping of medical reports (e.g., medical reports comprising similar vocabulary and syntactic patterns may be designated as a first category).

Method 1100 begins at operation 1102, wherein the patient information system extracts metadata from the medical report. In some embodiments, metadata may include one or more of timestamps, tags (e.g., report type, reporting physician, procedure code), patient demographic information (e.g., age, gender, BMI), diagnostic type (e.g., imaging modality used, assay parameters, etc.). In some embodiments, metadata may be stored in a DICOM header of a DICOM formatted file, wherein one or more tags relevant to the diagnostic scan may be recorded in the DICOM header.

At operation 1104, the patient information system, classifies the extracted metadata into one or more pre-determined categories. In some embodiments, the patient information system may include a lookup table or pivot table mapping one or more pieces of metadata to medical report categories. As an example, medical reports including metadata in the form of a tag for “tumor biopsy” may be classified as belonging to a first medical report category by querying a lookup table with the key “tumor biopsy”, wherein an associated value for said key is the medical report category corresponding to medical reports related to tumor biopsies, (i.e., the first category designation). In some embodiments, a medical report may include multiple pieces of metadata, and a medical report category may be determined for the medical report by taking a weighted average of the categories corresponding to each piece of metadata. In some embodiments, multiple pieces of metadata may each be mapped to a medical report category using a lookup table (e.g., a dictionary data structure) or a pivot table, and a most frequent medical report category may be selected as the medical report category to which the current medical report belongs.

At operation 1106, the patient information system maps the one or more pre-determined categories to at least a first entity recognition model in an entity recognition model library. Each entity recognition model stored in the entity recognition model library may be indexed by a medical report category of the pre-determined number of medical report categories, for which the entity recognition model has been trained (e.g., according to a training method employing a distinct set of training parameters). In some embodiments, a dictionary data structure or other implementation of a lookup table, may be used to efficiently map the medical report category of the current medical report to an entity recognition model trained to identify entities in medical reports belonging to said category. In some embodiments, the dictionary data structure or other lookup table may map medical report categories (in the form of a string or hash) to a location in non-transitory memory (e.g., the entity recognition model library) where the entity recognition model is stored, thereby enabling rapid access to the entity recognition model. Following operation 1106, method 1100 may end.

In this way, method 1100 enables rapid classification of a medical report into one or more of a pre-determined number of medical report categories based on the metadata attributes of the medical report, and further enables rapid access of an entity recognition model from a location in non-transitory memory. The entity recognition model so accessed may subsequently be used to identify named entities within the medical report, and a report summary may be generated based on the identified named entities.

Referring to FIG. 12, a flowchart of a second embodiment of a method 1200 for classifying a medical report into one of a pre-determined number of medical report categories is shown. Method 1200 may be executed by a patient information system, such as patient information system 100, or may be executed by a separate computing system before transmitting the medical report classification to the patient information system. Method 1200 may be executed alone, or as part of another method herein disclosed, such as at operation 306 of method 300, in order to determine a category or classification to which a currently evaluated medical report belongs.

Method 1200 begins at operation 1202, wherein the patient information system encodes the medical report as a feature vector. The patient information system may encode the medical report by converting attributes of the medical report into one or more numerical values, and populating a vector with the numerical values to produce the feature vector. The feature vector represents the medical report as a point in an N-dimensional space, wherein N is a positive integer greater than one which quantifies the number of distinct encoded features. In some embodiments, text of the medical report may be converted into one or more embedding vectors, (e.g., by an encoder such as a transformer model). In some embodiments, metadata may be included within the feature vector by encoding one or more pieces of the metadata as a numerical value, and populating the feature vector with the numerical value. As an example, the text of a report may be encoded as an embedding vector of dimension dₑ, and metadata of the report may be encoded as a second vector of dimension dₘ, the first and second vectors may be concatenated to produce a feature vector of dimension d_{f} wherein d_{f}=dₑ+dₘ. In some embodiments, categorical features of a medical report, such as scan type, report type, etc., may be encoded using one-hot encoding.

At operation 1204, the patient information system, classifies the medical report into one or more categories based on a proximity of the feature vector to one or more category clusters in a feature vector space. As discussed above in relation to operation 1202, the feature vector produced for a medical report may be considered as a point in an N-dimensional feature space (also referred to herein as a feature vector space), wherein each entry of the feature vector corresponds to a coordinate in the feature space. Thus, proximity between one or more previously encoded medical reports (e.g., medical reports of a training dataset, such as training dataset 402), and the medical report encoded at operation 1202, may occur by comparing the current feature vector with the feature vectors of one or more previously encoded medical reports. In some embodiments, the medical reports of a training dataset, such as training dataset 402 may each be encoded (e.g., according to operation 1202 described above), to produce a plurality of feature vectors of the training dataset.

A clustering algorithm, such as a K-means clustering algorithm, or other clustering algorithms known in the art, may be used segregate the medical reports of the training dataset into the pre-determined number of medical report categories/clusters, based on the encoded positions of the medical reports (i.e., the points in the feature space corresponding to the plurality of feature vectors). The identified clusters may, in some embodiments, be uniquely represented by an average or center point of the cluster, which itself may be represented as a point in the feature space. Thus, in some embodiments, operation 1204 may include the patient information system calculating distances between the current feature vector, and the identified clusters of the pre-determined number of medical report categories. In some embodiments, determining a distance between the current feature vector and a cluster may comprise determining a Euclidean distance between the current feature vector and a center of the cluster. In some embodiments, a cosine similarity may be used to determine proximity/similarity of the current feature vector with the identified clusters. The medical report may be classified as belonging to one or more of the pre-determined medical report categories based on the measured distances/similarities between the current feature vector and the previously identified clusters. In some embodiments, the medical report may be classified as belonging to a medical report category having a shortest distance and/or greatest similarity between a cluster corresponding to the medical report category and the current feature vector.

At operation 1206, the patient information system maps the one or more pre-determined categories to at least a first entity recognition model in an entity recognition model library. Each entity recognition model stored in the entity recognition model library may be indexed by a medical report category of the pre-determined number of medical report categories, for which the entity recognition model has been trained (e.g., according to a training method employing a distinct set of training parameters). In some embodiments, a dictionary data structure or other implementation of a lookup table, may be used to efficiently map the medical report category of the current medical report to an entity recognition model trained to identify entities in medical reports belonging to said category. In some embodiments, the dictionary data structure or other lookup table may map medical report categories (in the form of a string or hash) to a location in non-transitory memory (e.g., the entity recognition model library) where the entity recognition model is stored, thereby enabling rapid access to the entity recognition model. Following operation 1206, method 1200 may end.

In this way, method 1200 enables rapid classification of a medical report into one or more of a pre-determined number of medical report categories based on a feature vector encoding of the medical report, and further enables rapid access of an entity recognition model from a location in non-transitory memory. The entity recognition model so accessed may subsequently be used to identify named entities within the medical report, and a report summary may be generated based on the identified named entities.

Turning to FIG. 13, in the depicted embodiment, excerpt 1302 includes the word “tumor”, which has been labeled as an instance of the entity “cancer” by the entity recognition model. Specifically, the entity recognition model has inserted the markup tags <cancer> and </cancer> to identify the word “tumor” as cancer. A probability of the word “tumor” being accurately identified as “cancer” is also included, as described above. When generating the patient summary, a module of the patient summary system may search for the markup tags. When the markup tags are encountered, executable code of the module may replace the tagged entity with a graphical label, as shown in FIG. 14.

In FIG. 14, a summary display example 1400 shows an exemplary display excerpt 1402 generated from exemplary excerpt 1302 of model output example 1300 of FIG. 13, where display excerpt 1402 is displayed within a patient summary generated by a patient summary system (e.g., patient summary system 102), in accordance with an embodiment. Executable code of the patient summary system may detect markup tags in excerpt 1302 of FIG. 13, and insert a graphical label at a location of the markup tags. The graphical label may include formatting and/or highlighting such as, for example, a colored/shaded background, bold text, colored text, or other visual features to indicate the relevant entity. In some embodiments, the formatting and/or highlighting may be customized based on the probability value assigned by the entity recognition model.

Additionally, the formatting and/or highlighting may be specific to the entity. For example, a first entity recognition model may include a first formatting and/or highlighting for identifying a first entity; a second entity recognition model may include a second formatting and/or highlighting for identifying a second entity; and so on. In this way, when outputs of a plurality of entity recognition models are aggregated, each entity recognized by a respective entity recognition model may be indicated in a distinctive manner.

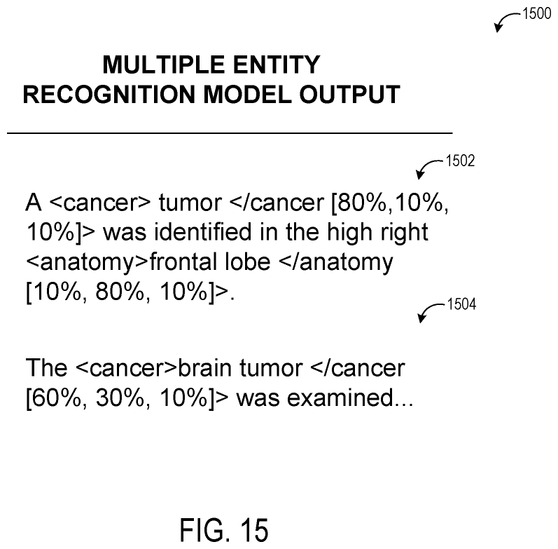

Similar to FIG. 13, FIG. 15 includes a model output example 1500 showing a first exemplary excerpt 1502 and a second exemplary excerpt 1504 of an output of a multiple entity recognition model, where the model output includes a probability vector including probability values for each entity on which the multiple entity recognition model is trained. The multiple entity recognition model is trained on two entities: “cancer”, and “anatomy”. For example, the multiple entity recognition model may be trained on a dataset including labeled cancer entities, and labeled anatomical parts.

As in FIG. 13, excerpt 1502 includes the word “tumor”, which has been labeled as an instance of the entity “cancer” by the entity recognition model. Specifically, the entity recognition model has inserted the markup tags <cancer> and </cancer> to identify the word “tumor” as cancer. A probability vector of the word “tumor” is also included, where the probability vector includes three probability values relating to the entities “cancer” and “anatomy”, and a probability of “tumor” not being identified as “cancer” or “anatomy”, in that order. A first probability value of 80% indicates a probability of “tumor” being identified as “cancer”. A second probability value of 10% indicates a probability of “tumor” being identified as “anatomy”. A third probability of 10% indicates a probability of “tumor” being identified as “outside” (e.g., a non-cancer and non-anatomy entity). As a result of the probability of “tumor” being identified as “cancer” being greater than the probability of “tumor” being identified as “anatomy” (or “outside”), the markup tags <cancer> and </cancer> are selected to label “tumor” as “cancer”.

Similarly, the expression “frontal lobe” has been labeled an instance of the entity “anatomy”, as a result of being assigned a greater probability than “cancer” and “outside” (e.g., 80% vs. 10% vs 10%).

In second display excerpt 1604, in accordance with the model output, the expression “brain tumor” has a 60% probability of being an instance of “cancer”, a 30% probability of being an instance of “anatomy”, and a 10% chance of being “outside”, as a result of the inclusion of the word “brain”. As a result of “brain tumor” having a higher probability of being an instance of “cancer” than being an instance of “anatomy”, the multiple entity recognition model includes the markup tags for “cancer”, while the probability vector includes information that “anatomy” is also a possibility.

In FIG. 16, a summary display example 1600 shows a first display excerpt 1602 and a second display excerpt 1604 generated from exemplary excerpts 1502 and 1504 of model output example 1500 of FIG. 15, where display excerpts 1602 and 1604 are displayed within a patient summary generated by a patient summary system (e.g., patient summary system 102), in accordance with an embodiment.

Exemplary display excerpt 1602 may be displayed on a screen to a caregiver (e.g., on care provider device 134). When generating the patient summary, a module of the patient summary system may search for the markup tags for “cancer” and “anatomy”. When the markup tags are encountered, executable code of the module may replace a respective tagged entity with a respective graphical label. As described in reference to FIG. 14, the graphical label may include formatting and/or highlighting such as, for example, a colored/shaded background, bold text, colored text, or other visual features to indicate the relevant entity. The formatting and/or highlighting may be customized based on the probability value assigned by the entity recognition model.

In second display excerpt 1604, labels for “cancer” and “anatomy” may be both included for the word “brain tumor”, due to a difference between the probabilities (e.g., 60%, 30%, 10%, from FIG. 15) being below a threshold difference. Additionally, the labels for “cancer” and “anatomy” may be visually distinguished from each other based on the difference in probabilities. For example, the label “cancer” may be displayed with a first formatting (e.g., in white), and the label “anatomy” may be displayed with a second formatting (e.g., in a darker shade). In this way, an uncertainty of the model output may be communicated to the caregiver. It should be appreciated that in other embodiments, different types of labeling techniques and/or different types of formatting and/or highlighting may be used.

Thus, methods and systems are provided for a patient summary system for summarizing patient information in digitized medical reports, for example, of an EMR of a patient, based on an identification of named entities within the digitized medical reports. The medical reports may be classified into one of a plurality of pre-determined categories, and an entity recognition model trained for said category may be accessed and employed to identify named entities in the medical reports. The patient summary system may then refine the identified named entities to produce a summary of the medical reports, wherein in some embodiments the summary may comprise one or more excerpts from the medical reports visually emphasizing one or more of the identified named entities. In some examples, the caregiver may specify one or more categories of named entities that they are interested in, and the patient summary system may generate a summary specific to those entities. By viewing the summary rather than reviewing the medical report, the caregiver may save time, allowing the caregiver to find information more quickly. By not having to review a plurality of medical reports in an EMR when seeking patient information, an efficiency of the caregiver and an amount of time the caregiver has to attend to other tasks may be increased. Further, the labeled excerpts may be formatted using labels of differing colors, shading, highlighting, formatting, or other features such that the caregiver may quickly scan for informative named entities, saving the caregiver additional time.

By intelligently setting training parameters based on domain knowledge and imbalances in a training dataset, a plurality of named entity recognition models may be produced which specialize in identifying informative named entities in corresponding categories of medical reports. Thus, using a single training dataset, an entity recognition model library may be produced which, on aggregate, more accurately identifies salient information in medical reports, without a need to increase a size of the training dataset. Further, by adapting the loss based on a plurality of target classes, to increase a model's prioritization of learning for target classes, instead of dropping or pooling named entities outside of the target classes, a more robust and context aware representation may be learned by the name entity recognition models.

The technical effect of adjusting losses for a plurality of named entity classes based on a medical report category and a frequency of the named entity annotations in the training dataset is that an entity recognition model may be produced which has a reduced probability of mis-labeling named entities carrying relevant information for the report category, even when said named entities belong to an under-represented category of named entities within the training dataset. Further, by scaling losses for a plurality of named entity classes, as opposed to dropping or pooling named entity classes to force a model to prioritize one or more target classes, a probability of overfitting may be reduced, and an efficiency of knowledge extraction from the training dataset may be improved.

The disclosure also provides support for a method comprising: receiving a medical report for a patient, classifying the medical report into a category of a plurality of pre-determined categories, matching the medical report with an entity recognition model from a library of entity recognition models based on the category, identifying a plurality of named entities in the medical report using the entity recognition model, refining the plurality of named entities to produce a summary of the medical report, and displaying the summary of the medical report via a display device. In a first example of the method, the library of entity recognition models comprises a plurality of machine learning models trained using a same training dataset, and wherein each of the plurality of machine learning models was trained using a distinct set of training parameters. In a second example of the method, optionally including the first example, the plurality of machine learning models includes at least one trained entity recognition model for each of the plurality of pre-determined categories, and wherein the distinct set of training parameters associated with each of the plurality of machine learning models is determined based on a category of the plurality of pre-determined categories. In a third example of the method, optionally including one or both of the first and second examples, the distinct set of training parameters for the entity recognition model comprises a set of loss adjustment factors for a list of target entity classes, wherein the set of loss adjustment factors and the list of target entity classes for the entity recognition model are determined based on the category of the medical report. In a fourth example of the method, optionally including one or more or each of the first through third examples, the set of loss adjustment factors for the list of target entity classes are each greater than one. In a fifth example of the method, optionally including one or more or each of the first through fourth examples, the distinct set of training parameters for the entity recognition model comprises a set of loss adjustment factors for one or more non-target entity classes, and wherein the set of loss adjustment factors for the one or more non-target entity classes is equal to or less than one. In a sixth example of the method, optionally including one or more or each of the first through fifth examples, matching the medical report with the entity recognition model from the library of entity recognition models comprises: extracting metadata from the medical report, classifying the medical report into the category of the plurality of pre-determined categories, and mapping the category to the entity recognition model in the library of entity recognition models. In a seventh example of the method, optionally including one or more or each of the first through sixth examples, matching the medical report with the entity recognition model from the library of entity recognition models comprises: encoding the medical report as a feature vector, classifying the medical report into the category of the plurality of pre-determined categories based on a proximity of the feature vector to one or more category clusters in a feature vector space, and mapping the category to the entity recognition model in the library of entity recognition models.

The disclosure also provides support for a method comprising: selecting a category from a plurality of pre-determined medical report categories, determining a plurality of training parameters based on the category, selecting a training data pair, wherein the training data pair comprises a medical report and a list of ground truth entity annotations, mapping the medical report to a list of entity classifications using an entity recognition model, determining a base loss for each entity classification in the list of entity classifications by comparing each entity classification with a corresponding ground truth entity annotation from the list of ground truth entity annotations, adjusting the base loss for each entity classification based on the plurality of training parameters to produce a list of adjusted losses, updating parameters of the entity recognition model based on the list of adjusted losses, and storing the entity recognition model in an entity recognition model library. In a first example of the method, the plurality of training parameters includes a list of target entity classes and a corresponding list of loss adjustment factors, and wherein adjusting the base loss for each entity classification based on the plurality of training parameters, comprises: determining if the corresponding ground truth entity annotation matches a target class from the list of target entity classes, responding to the corresponding ground truth entity annotation matching the target class by: selecting a loss adjustment factor from the list of loss adjustment factors based on the target class, and scaling the base loss by the loss adjustment factor to produce an adjusted loss. In a second example of the method, optionally including the first example, each entity classification comprises a vector of entity classification scores for each of a plurality of entity classes, wherein the plurality of training parameters includes a list of target entity classes and a corresponding list of loss adjustment factors, and wherein adjusting the base loss for each entity classification based on the plurality of training parameters, comprises: determining if an entity classification score from the vector of entity classification scores matches a target class from the list of target entity classes, responding to the entity classification score matching the target class by: selecting a loss adjustment factor from the list of loss adjustment factors based on the target class, and scaling the base loss for the entity classification score by the loss adjustment factor to produce an adjusted loss. In a third example of the method, optionally including one or both of the first and second examples, mapping the medical report to a list of entity classifications using the entity recognition model comprises: tokenizing the medical report to produce a plurality of tokens, encoding the plurality of tokens as a plurality of embedding vectors, and mapping each of the plurality of embedding vectors to a corresponding entity classification to produce the list of entity classifications. In a fourth example of the method, optionally including one or more or each of the first through third examples, storing the entity recognition model in the entity recognition model library includes indexing the entity recognition model according to the category from the plurality of pre-determined medical report categories. In a fifth example of the method, optionally including one or more or each of the first through fourth examples, the plurality of training parameters includes a loss adjustment factor vector, and wherein adjusting the base loss for each entity classification based on the plurality of training parameters, comprises: selecting a loss adjustment factor from the loss adjustment factor vector based on the corresponding ground truth entity annotation, and scaling the base loss by the loss adjustment factor to produce an adjusted loss. In a sixth example of the method, optionally including one or more or each of the first through fifth examples, each entity classification comprises a vector of entity classification scores for each of a plurality of entity classes, wherein the plurality of training parameters includes a loss adjustment factor vector, and wherein adjusting the base loss for each entity classification based on the plurality of training parameters, comprises: scaling the base loss for each entity classification score in the vector of entity classification scores by a corresponding loss adjustment factor from the loss adjustment factor vector, to produce an adjusted loss. In a seventh example of the method, optionally including one or more or each of the first through sixth examples, the base loss is a base loss vector comprising a plurality of losses for the plurality of entity classes, and wherein scaling the base loss for each entity classification score in the vector of entity classification scores by the corresponding loss adjustment factor from the loss adjustment factor vector comprises taking a dot product of the base loss vector and the loss adjustment factor vector.

The disclosure also provides support for a system for automatically summarizing medical reports, the system comprising: an electronic medical records database, and a patient summary system communicatively coupled to the electronic medical records database, the patient summary system comprising: instructions stored in non-transitory memory of the patient summary system, and a processor, that when executing the instructions causes the patient summary system to: access a medical report for a patient from the electronic medical records database, classify the medical report into a category of a plurality of pre-determined categories, match the medical report with an entity recognition model from a library of entity recognition models, identify a plurality of named entities in the medical report using the entity recognition model, refine the plurality of named entities to produce a summary of the medical report, and display the summary of the medical report via a display device. In a first example of the system the system further comprising a care provider device communicatively coupled to the patient summary system, and wherein the processor is configured to display the summary of the medical report via the display device by: transmitting the summary of the medical report to the care provider device, wherein the care provider device includes the display, and displaying the summary of the medical report via the display device of the care provider device. In a second example of the system, optionally including the first example, the library of entity recognition models comprises a plurality of machine learning models trained using a same training dataset, and wherein each of the plurality of machine learning models was trained using a distinct set of training parameters. In a third example of the system, optionally including one or both of the first and second examples, the distinct set of training parameters for the entity recognition model comprises a set of loss adjustment factors for a list of target entity classes, wherein the set of loss adjustment factors and the list of target entity classes for the entity recognition model are determined based on the category of the medical report.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method comprising:
receiving a medical report for a patient;
classifying the medical report into a category of a plurality of pre-determined categories;
matching the medical report with an entity recognition model from a library of entity recognition models based on the category;
identifying a plurality of named entities in the medical report using the entity recognition model;
refining the plurality of named entities to produce a summary of the medical report; and
displaying the summary of the medical report via a display device.

2. The method of claim 1, wherein the library of entity recognition models comprises a plurality of machine learning models trained using a same training dataset, and wherein each of the plurality of machine learning models was trained using a distinct set of training parameters.

3. The method of claim 2, wherein the plurality of machine learning models includes at least one trained entity recognition model for each of the plurality of pre-determined categories, and wherein the distinct set of training parameters associated with each of the plurality of machine learning models is determined based on a category of the plurality of pre-determined categories.

4. The method of claim 2, wherein the distinct set of training parameters for the entity recognition model comprises a set of loss adjustment factors for a list of target entity classes, wherein the set of loss adjustment factors and the list of target entity classes for the entity recognition model are determined based on the category of the medical report.

5. The method of claim 4, wherein the set of loss adjustment factors for the list of target entity classes are each greater than one.

6. The method of claim 2, wherein the distinct set of training parameters for the entity recognition model comprises a set of loss adjustment factors for one or more non-target entity classes, and wherein the set of loss adjustment factors for the one or more non-target entity classes is equal to or less than one.

7. The method of claim 1, wherein matching the medical report with the entity recognition model from the library of entity recognition models comprises:
extracting metadata from the medical report;
classifying the medical report into the category of the plurality of pre-determined categories; and
mapping the category to the entity recognition model in the library of entity recognition models.

8. The method of claim 1, wherein matching the medical report with the entity recognition model from the library of entity recognition models comprises:
encoding the medical report as a feature vector;
classifying the medical report into the category of the plurality of pre-determined categories based on a proximity of the feature vector to one or more category clusters in a feature vector space; and
mapping the category to the entity recognition model in the library of entity recognition models.

9. A method comprising:
selecting a category from a plurality of pre-determined medical report categories;
determining a plurality of training parameters based on the category;
selecting a training data pair, wherein the training data pair comprises a medical report and a list of ground truth entity annotations;
mapping the medical report to a list of entity classifications using an entity recognition model;
determining a base loss for each entity classification in the list of entity classifications by comparing each entity classification with a corresponding ground truth entity annotation from the list of ground truth entity annotations;
adjusting the base loss for each entity classification based on the plurality of training parameters to produce a list of adjusted losses;
updating parameters of the entity recognition model based on the list of adjusted losses; and
storing the entity recognition model in an entity recognition model library.

10. The method of claim 9, wherein the plurality of training parameters includes a list of target entity classes and a corresponding list of loss adjustment factors, and wherein adjusting the base loss for each entity classification based on the plurality of training parameters, comprises:
determining if the corresponding ground truth entity annotation matches a target class from the list of target entity classes;
responding to the corresponding ground truth entity annotation matching the target class by:
selecting a loss adjustment factor from the list of loss adjustment factors based on the target class; and
scaling the base loss by the loss adjustment factor to produce an adjusted loss.

11. The method of claim 9, wherein each entity classification comprises a vector of entity classification scores for each of a plurality of entity classes, wherein the plurality of training parameters includes a list of target entity classes and a corresponding list of loss adjustment factors, and wherein adjusting the base loss for each entity classification based on the plurality of training parameters, comprises:
determining if an entity classification score from the vector of entity classification scores matches a target class from the list of target entity classes;
responding to the entity classification score matching the target class by:
selecting a loss adjustment factor from the list of loss adjustment factors based on the target class; and
scaling the base loss for the entity classification score by the loss adjustment factor to produce an adjusted loss.

12. The method of claim 9, wherein mapping the medical report to a list of entity classifications using the entity recognition model comprises:
tokenizing the medical report to produce a plurality of tokens;
encoding the plurality of tokens as a plurality of embedding vectors; and
mapping each of the plurality of embedding vectors to a corresponding entity classification to produce the list of entity classifications.

13. The method of claim 9, wherein storing the entity recognition model in the entity recognition model library includes indexing the entity recognition model according to the category from the plurality of pre-determined medical report categories.

14. The method of claim 9, wherein the plurality of training parameters includes a loss adjustment factor vector, and wherein adjusting the base loss for each entity classification based on the plurality of training parameters, comprises:
selecting a loss adjustment factor from the loss adjustment factor vector based on the corresponding ground truth entity annotation; and
scaling the base loss by the loss adjustment factor to produce an adjusted loss.

15. The method of claim 9, wherein each entity classification comprises a vector of entity classification scores for each of a plurality of entity classes, wherein the plurality of training parameters includes a loss adjustment factor vector, and wherein adjusting the base loss for each entity classification based on the plurality of training parameters, comprises:
scaling the base loss for each entity classification score in the vector of entity classification scores by a corresponding loss adjustment factor from the loss adjustment factor vector, to produce an adjusted loss.

16. The method of claim 15, wherein the base loss is a base loss vector comprising a plurality of losses for the plurality of entity classes, and wherein scaling the base loss for each entity classification score in the vector of entity classification scores by the corresponding loss adjustment factor from the loss adjustment factor vector comprises taking a dot product of the base loss vector and the loss adjustment factor vector.

17. A system for automatically summarizing medical reports, the system comprising:
an electronic medical records database; and
a patient summary system communicatively coupled to the electronic medical records database, the patient summary system comprising:
instructions stored in non-transitory memory of the patient summary system; and
a processor, that when executing the instructions causes the patient summary system to:
access a medical report for a patient from the electronic medical records database;
classify the medical report into a category of a plurality of pre-determined categories;
match the medical report with an entity recognition model from a library of entity recognition models;
identify a plurality of named entities in the medical report using the entity recognition model;
refine the plurality of named entities to produce a summary of the medical report; and
display the summary of the medical report via a display device.

18. The system of claim 17, the system further comprising a care provider device communicatively coupled to the patient summary system, and wherein the processor is configured to display the summary of the medical report via the display device by:
transmitting the summary of the medical report to the care provider device, wherein the care provider device includes the display; and
displaying the summary of the medical report via the display device of the care provider device.

19. The system of claim 17, wherein the library of entity recognition models comprises a plurality of machine learning models trained using a same training dataset, and wherein each of the plurality of machine learning models was trained using a distinct set of training parameters.

20. The system of claim 19, wherein the distinct set of training parameters for the entity recognition model comprises a set of loss adjustment factors for a list of target entity classes, wherein the set of loss adjustment factors and the list of target entity classes for the entity recognition model are determined based on the category of the medical report.
